(19) United States
(12) Patent Application Publication
(10) Pub. No.: US 2013/0030339 A1
(43) Pub. Date: Jan. 31, 2013

(54) GAUZE ASSEMBLY FOR REINFORCING TISSUE WHICH EXTENDS AROUND A SURGICALLY CREATED STOMA

(71) Applicant: Johan De Gols, Heusden-Zolder (BE)

(72) Inventor: Johan De Gols, Heusden-Zolder (BE)

(21) Appl. No.: 13/638,414

(22) PCT Filed: Mar. 31, 2011
(86) PCT No.: PCT/IB2011/051386

§ 371 Date: Sep. 28, 2012

(30) Foreign Application Priority Data
Mar. 31, 2010 (BE) 2010/0206

(51) Int. Cl. A61F 13/00

(52) U.S. Cl. 602/41

(57) ABSTRACT

Gauze assembly for reinforcing a stoma, comprising a reinforcing gauze with an ingrowth side and an intestinal side so that the reinforcing gauze can grow into a tissue on one side, which reinforcing gauze comprises a stoma opening with flaps which extend substantially towards the centre point of the stoma opening, which reinforcing gauze furthermore comprises a stoma access path, wherein the gauze assembly furthermore comprises a handling gauze connected with the intestinal side of said flaps so that said handling gauze can be positioned in a substantially tubular form through said stoma opening and hence said flaps can be folded at least partly towards the ingrowth side.

The invention relates to a gauze assembly for reinforcing tissue which extends around a surgically created stoma, wherein said gauze assembly comprises a biocompatible reinforcing gauze with a first side and a second side such that said reinforcing gauze can grow into said tissue, which reinforcing gauze comprises in a central zone a stoma opening, which stoma opening has an edge formed by flaps which extend from said reinforcing gauze substantially towards a centre point of said stoma opening, which reinforcing gauze furthermore comprises a stoma access path which extends from said stoma opening to an outermost edge of the reinforcing gauze.

A stoma is an artificial, surgically created opening in the bowel at the abdominal wall. Less frequently it is a synthetic opening through the abdominal wall to which a urinary catheter is connected. A stoma is necessary if a substantial part of the bowel has been surgically removed, for example due to cancer or infection, whereby normal discharge of the bowel content through the large intestine and anus is no longer possible. When a stoma is created, an opening is made in the abdominal wall.

By creating an opening in the abdominal wall, weakening occurs. Due to this weakening a hernia can result up to years after creation of the stoma. A hernia is a protrusion of an organ or tissue from a body cavity in which it normally lies. On a parastomal hernia, referred to below as a parastomal hernia, the intestines bulge out adjacent to the opening in the abdominal wall through the abdominal flesh and the muscle tissue. This can cause squeezing of the intestines. A parastomal hernia can be repaired by reinforcing the tissue around the stoma. For this a gauze is attached to the inside of the abdominal wall.

A parastomal hernia typically occurs some time after creation of the stoma when the bowel which extends through the abdominal wall has grown onto the abdomen skin. To minimise cutting and wound creation, a parastomal hernia is repaired by laparoscopy whereby typically carbon dioxide gas is introduced into the abdominal cavity in order to inflate the abdomen so that a working space is created between the intestines and the abdominal wall tissue. A further advantage is that with the use of this laparoscopic method, contact between the stoma opening and the gauze assembly can be prevented because the gauze assembly is not brought into the inside of the abdomen close to the stoma opening. Such contact would cause contamination of the gauze and hence infection.

Use of this laparoscopic method has the consequence that the gauze must be positioned and attached to the inside of the abdominal wall from the inside. Furthermore this has the consequence that for handling, in particular when the gauze is substantially positioned, the only elements which are visible and able to be handled lie on the inside of the gauze. Elements which are on the outside of the gauze, for example between the gauze and the abdominal wall, will not be visible or able to be handled because, viewed from the outside, the gauze sits in front.

A gauze for reinforcing tissue which extends around a surgically created stoma is known from US 2008/0167729 which is regarded as the closest prior art. This known reinforcing gauze comprises at its stoma opening flaps which extend towards the centre of the opening. When the reinforcing gauze is used to repair a parastomal hernia, these flaps are folded inwards so that they lie against the bowel which protrudes through the abdominal wall.

One problem with the reinforcing gauze is because the flaps are folded towards the inside, these flaps do not lie positioned anatomically correctly in the abdominal wall. This incorrect positioning will in the short term have few or no consequences because in the short term the body can be regarded as a static element. However in the long term the body will typically deform by ageing and/or a change of habits of the person. If the reinforcing gauze is not positioned anatomically correctly, the deformation of the body will be countered by the reinforcing gauze, causing strains in the body. These strains in a further phase can lead to complications which require medical intervention. However folding the flaps towards to the back so that the reinforcing gauze is correctly positioned is not possible because this side cannot be accessed via laparoscopic treatment. Namely viewed from the working space in the inflated abdomen, the gauze sits between the handling elements and/or camera and the back, whereby this side is not accessible and not visible.

It is an object of the invention to provide a gauze assembly for reinforcing tissue around a stoma which can be positioned anatomically correctly via laparoscopic treatment.

For this a gauze assembly according to the invention has the feature that said gauze assembly furthermore comprises a biocompatible handling gauze which is connected with the second side of said flaps in such a manner that said handling gauze can be positioned substantially tubular through said stoma opening, whereby said flaps can at least partly be folded in the direction towards the said first side. The handling gauze which is positionable tubular through the stoma opening can thus be placed around the bowel which extends through the abdominal wall. The handling gauze is connected with said flaps whereby the flaps, via the handling gauze, can be positioned in relation to the bowel. As the handling gauze extends through the stoma opening, the handling gauze is visible and accessible on both sides of the reinforcing gauze. Manipulation of the handling gauze on one side of the reinforcing gauze will result in a movement of the handling gauze on the other side of the reinforcing gauze. Thus the handling gauze can be positioned on the other side of the reinforcing gauze from the side on which it is effectively manipulated. This allows laparoscopic positioning, via the working cavity in the abdomen, of the handling gauze on the back behind the reinforcing gauze. Because the flaps are connected with the handling gauze, the flaps are also positionable and can be folded towards the back. Thus the flaps lie anatomically correctly in the abdominal wall and deformations in the body can be absorbed. One surprising effect occurs on use of the invention. Because the flaps are connected with the handling gauze and the handling gauze is positioned and connected with the bowel which extends through the abdominal wall, the individual flaps need not be connected with this bowel. Only one relatively large surface, the handling gauze, must be correctly connected with the bowel, according to the invention, whereas in the prior art every flap which has a relatively small surface must be correctly connected with the bowel. Not only do correct positioning and connection of a flap with the bowel require far more precision due to the small surface of the flap, but due to the quantity of flaps which must be individually connected with the bowel, the positioning of the gauze according to the prior art is extremely labour intensive and consequently requires a lot of time. Positioning and connecting of the handling gauze with the bowel is much easier and requires far less precision and takes substantially less time. This is a surprising advantage of the invention in relation to the prior art.

Preferably said first side is an ingrowth side and said second side is the intestinal side. An ingrowth side will cause tissue irritation because the reinforcing gauze grows on this ingrowth side. An intestinal side is intended to cause as little tissue irritation as possible so that no perforation of the intestines occurs due to irritation. The consequence of an ingrowth and an intestinal side is that the reinforcing gauze will ingrow on one side.

One problem with use of such a reinforcing gauze according to the prior art is that because the flaps are folded towards the inside, these flaps come to lie with the ingrowth side, and not with the intestinal side, against the bowel. The ingrowth of such an ingrowth side of a reinforcing gauze in a bowel can result in perforations of the bowel. As a result contamination of the abdominal cavity can occur, with the associated complications.

Because the reinforcing gauze according to the invention is designed to have the flaps fold towards the back i.e. towards the ingrowth side, it is not the ingrowth side but the intestinal side of the reinforcing gauze which is directed towards the bowel. This intestinal side is intended to irritate the bowel as little as possible and thus minimise the chances of perforation of the bowel.

Preferably said handling gauze has at least one substantially straight edge whereby said flaps are connected with said handling gauze at said edge. Because the handling gauze can be positioned through the stoma opening and the flaps are connected with the handling gauze at the level of its straight edge, the handling gauze in its fitted position protrudes minimally on the ingrowth side of the reinforcing gauze.

As a result the tubular part of the handling gauze which is positioned, viewed laparoscopically, behind the reinforcing gauze is of minimal length.

Consequently it is easier to position this laparoscopically.

Preferably a first flap of said first flaps adjacent to said stoma access path is connected at one end of said straight edge. Thus the stoma access path firstly and access to the tubular handling gauze secondly are aligned. This simplifies positioning of the gauze assembly around the bowel which extends through the abdominal wall.

Preferably a second flap of said flaps adjacent to said stoma access path is connected at a distance from another end, opposite said first end, of said straight edge so that on tubular positioning of said handling gauze, an overlap of the handling gauze occurs. By overlap the correct positioning of the handling gauze is simplified, namely the two ends are attached overlaid and if desired mutually connected, whereby the handling gauze cannot extend other than in a tubular fashion. A further advantage of this overlap is that on positioning, the stoma access path can be held open whereby, laparoscopically viewed, the back of the reinforcing gauze is accessible at this stoma access path. The overlap of the handling gauze takes place at the stoma access path. Thus the overlap can be positioned and attached with the bowel, viewed laparoscopically, behind the reinforcing gauze. This allows the handling gauze to be positioned very precisely in relation to and be connected with the bowel which extends through the abdominal wall, even on the ingrowth side of the reinforcing gauze.

Preferably each of said flaps comprises a flap end which is as close as possible to said centre point, said flaps being connected with said handling gauze at their flap ends. In the fitted state the handling gauze lies against the bowel which extends through the abdominal wall. This has the result that also the flaps which are connected with the handling gauze lie against the bowel. Such a bowel is elastic and its diameter can vary. Because the flaps ends which are folded in the positioned state are connected with the handling gauze, diameter variations can be absorbed. Namely if the flaps are folded more towards the inside, they are closer to the centre point of the stoma opening. Also when folded more strongly, the flap ends are further away from the centre point.

Preferably said handling gauze is made of a material which is totally decomposable by tissue. Thus the handling gauze does not have an ingrowth side but is completely broken down and no material ingrows. The bowel which is in contact with the handling gauze cannot be damaged by the ingrowth material, whereby no perforations can occur. The break-down of a material is accompanied by tissue irritation whereby scar tissue is formed. Because the flap ends are connected with the handling gauze, formation of scar tissue on the bowel as a result of the handling gauze causes the scar tissue to grow both with the bowel and with the flap ends. This is despite the fact that the bowel lies on the intestinal side of the flaps which in normal circumstances would not lead to growth. As a result there is a connection between the reinforcing gauze and the bowel whereby further parastomal hernias in the future are prevented.

The invention will now be described in more detail with reference to embodiment examples shown in the drawing.

The drawing shows:

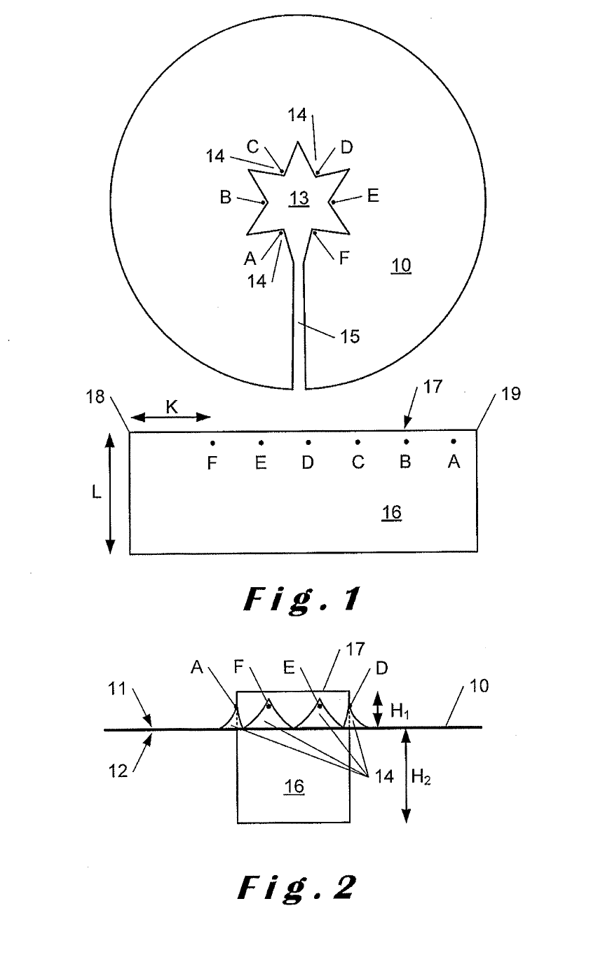

FIG. 1 a reinforcing gauze and a handling gauze from the gauze assembly according to the invention;

FIG. 2 a side view of a gauze assembly according to the invention;

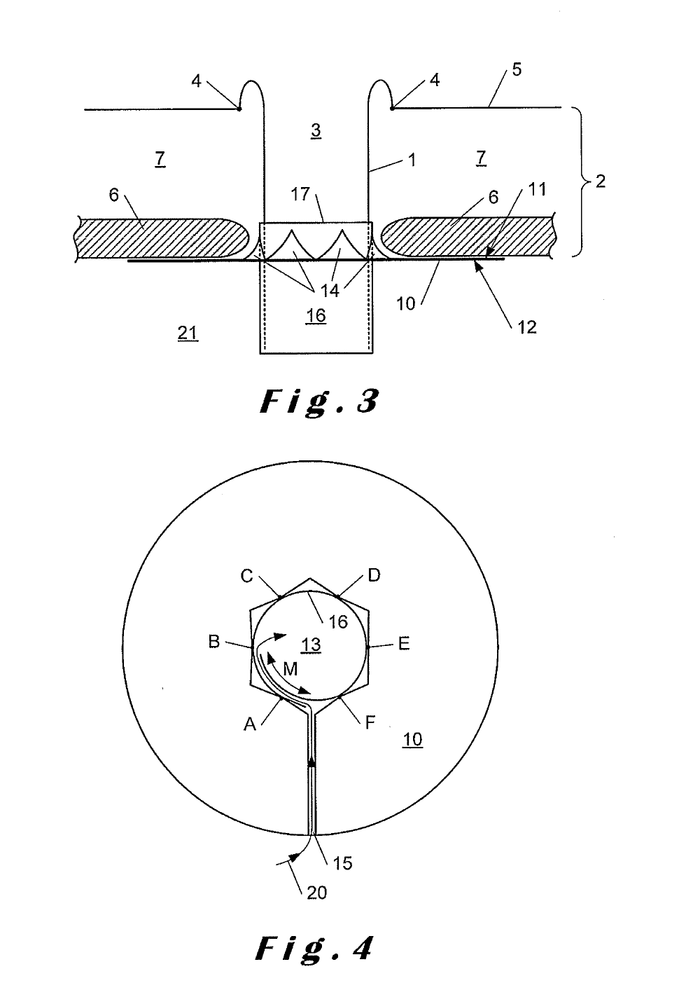

FIG. 3 a side view of a gauze assembly according to the invention placed around a stoma; and

FIG. 4 a top view of a gauze assembly according to the invention.

In the drawing the same or similar elements have the same reference numerals.

A stoma is an artificial opening to discharge faeces and urine. The invention will be explained in relation to a stoma for faeces or a bowel stoma, referred to below as a stoma. It will however be clear that the invention can also be used for stomas for urine. Similarly the invention is also applicable to the repair of a hernia adjacent to the oesophagus through the midriff, called a para-oesophagal hernia. Furthermore the invention is also applicable to the repair of a hernia in the small pelvis. The invention is therefore not restricted to gauze assemblies for reinforcing tissue around intestinal stomas. If such a gauze assembly is used to reinforce tissue in the body, this gauze assembly will preferably be positioned anatomically correctly. Anatomically correctly means that the gauze assembly follows the surface curvature of the tissue against which it is placed. If the surface curvature of a tissue is not followed by a gauze assembly, in other words if the gauze assembly is not positioned anatomically correctly, deformations of this tissue cannot be absorbed by the gauze assembly and strains occur between the gauze assembly and the tissue. In the pelvic floor where in a natural situation the bowel is connected with the outside of the body, the muscles around the bowel lie in a funnel shape towards the outside. Therefore the gauze assembly must be formed funnel-like towards the outside in order, in analogy with the pelvic floor, to lie anatomically correctly. For correct functioning of the body, each gauze assembly must be placed anatomically correctly in the body.

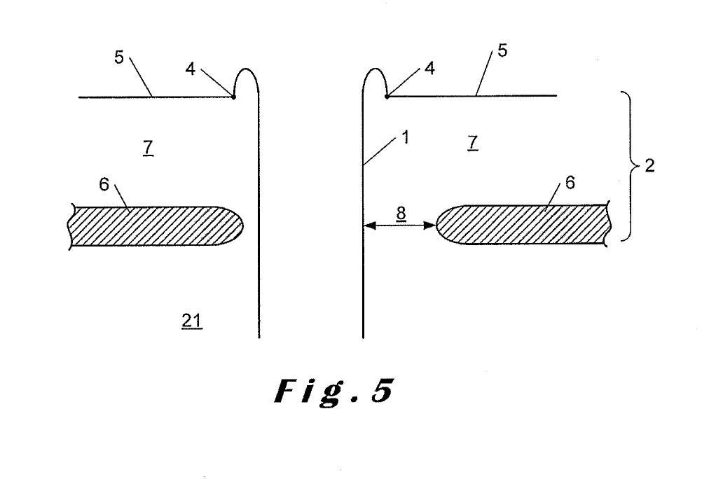

A stoma is an outlet 3 from the bowel 1 through the abdominal wall 2 as shown in FIG. 5. Such an outlet is created when the faeces cannot leave the body via the natural path. When the faeces leave the stoma, they are captured via known capture means and known methods for capturing faeces. The bowel grows via edges at its open end onto the skin 5 of the abdomen as shown with number 4. Thus there is no opening adjacent to the bowel at the site of the skin 5, from the inside to the outside and conversely.

The abdominal wall 2 furthermore contains muscle tissue 6 and fatty tissue 7, which fatty tissue 7 is substantially located between the muscle tissue 6 and the skin 5. The muscle tissue 6 and the fatty tissue 7, in contrast to the skin 5, are not directly connected with the bowel 1. Via the elongation and stretching of the muscle tissue 6 and the fatty tissue 7 over time, a substantial opening 8 can form between the muscle tissue and/or the fatty tissue 7 on one side and the bowel 1 on the other. Intestines can move through this opening 8, coming to sit between the muscle tissue 6 and/or fatty tissue 7 on one side and the skin 5 on the other, known as a parastomal hernia. Such a parastomal hernia has several disadvantages, including the risk that the bowel will be squeezed and the aesthetic and associated psychological effect on the patient, including deformation of the abdominal wall whereby the capture means connected to the abdomen at the site of the stoma opening can detach. The invention provides a gauze assembly with which such a parastomal hernia can be repaired. The gauze assembly according to the invention can also be used to prevent a parastomal hernia. Such a gauze assembly can be fitted when creating a stoma to prevent future parastomal hernia.

The gauze assembly according to the invention comprises a reinforcing gauze 10 with which tissue can be reinforced. The reinforcing gauze 10 is made of biocompatible material and comprises a reinforcing mesh which is intended to grow into the tissue on one side. The reinforcing gauze 10 is intended to grow with the abdominal wall 2, in particular the muscle tissue 6. The reinforcing gauze 10 is also intended to not grow into the intestine, and to irritate the latter as little as possible. Thus the reinforcing gauze 10 has an ingrowth side 11 and an intestinal side 12, whereby the reinforcing gauze 10 is intended to ingrow and strengthen the tissue on the side of its ingrowth side 11, and is intended to not grow into the bowel on its intestinal side. One example of a gauze suitable for use as a reinforcing gauze according to the invention is the surgical gauze called PROCEED® sold by Ethicon Products. However according to the invention a reinforcing gauze can also be used which is intended to irritate tissue as little as possible on both sides, i.e. a tissue without predefined ingrowth side.

The dimensions and shape of the reinforcing gauze 10 are less relevant to the invention. Thus the reinforcing gauze 10 in the figures is shown in circular form.

However a square or triangular reinforcing gauze 10 can also be used in the invention. The reinforcing gauze 10 is fitted with an opening 13 in a central region of the reinforcing gauze 10. This opening 13 is provided such that the bowel 1 can protrude through this, whereby this opening 13 is also known as the stoma opening 13.

The opening 13 in the reinforcing gauze 10 has an edge formed by flaps 14 which extend from the reinforcing gauze towards a centre point of the opening 13. The flaps 14 extend from the reinforcing gauze 10 and preferably form a whole with the reinforcing gauze 10. The flaps 14 are preferably sawtooth-like.

The flaps 14 preferably have a flap end which lies as close as possible to the centre point of the opening 13. The flap ends of the flaps 14 are indicated in the figures with A, B, C, D, E and F. The number of flaps 14 depends on the diameter of the opening 13. Preferably the opening is surrounded by 3 to 17 flaps, more preferably by 5 to 10 flaps, most preferably by 6 to 8 flaps.

The reinforcing gauze 10 furthermore has an access path 15 through which the gauze can be positioned around the bowel. This access path 15 is also known as the stoma access path 15. This access path 15 allows the reinforcing gauze 10 to be placed around a bowel which is connected on one side with the abdominal cavity and with the other side has grown into the skin of the abdomen. The access path 15 extends from the opening 13 to the peripheral edge of the reinforcing gauze 10.

The gauze assembly according to the invention furthermore comprises a handling gauze 16. The handling gauze 16 is made of a biocompatible material. Preferably the handling gauze 16 has no reinforcing mesh. The handling gauze 16 is preferably intended not to ingrow but to be dissolved completely over time because it is made of a material decomposable by tissue. The break-down of the material by tissue causes irritation of the tissue, whereby a slight quantity of scar tissue results. The handling gauze 16 intended to be positioned around the bowel therefore preferably stimulates the formation of scar tissue on this bowel. One example of a gauze suitable for use as a handling gauze 16 according to the invention is a surgical gauze called VICRYL® sold by Ethicon Products.

The handling gauze 16 preferably comprises at least one straight edge 17 with an end 18 and a further end 19 located opposite the end 18. The handling gauze 16 is intended to be positioned in tubular form through the stoma opening 13 of the reinforcing gauze 10. For this the handling gauze 13 can be rolled up, producing a tubular form with a diameter which is smaller than or equal in size to the diameter of the stoma opening 13 so that the tubular form passes through it.

According to the invention the handling gauze 16 is connected with the flaps 14 such that the handling gauze 16 can be positioned in tubular form through the stoma opening 13. A handling gauze 16 positioned in tubular form through the stoma opening 13 of the reinforcing gauze 10 is shown in FIG. 2 in side view and in FIG. 4 in top view. The flaps 14 are connected with the handling gauze 16 lying with their intestinal side 12 against one side of the handling gauze 16. Preferably the flaps 14 at their flap ends are connected with the handling gauze 16. The FIG. 1 shows for illustration points A to F on both the reinforcing gauze 10 and the handling gauze 16 to illustrate how these are connected together. By connecting points A together, points B together, points C together, points D together, points E together and points F together, the handling gauze 16 is connected with the flaps 14 of the reinforcing gauze 10 so that the handling gauze 16 can be positioned in tubular form through the stoma opening 13. FIGS. 2 and 4 also show at least part of points A to F in a position where the reinforcing gauze 10 is connected with the handling gauze 16.

The handling gauze 16 can be connected with the reinforcing gauze 10 in various ways. The connection is made biocompatible by the use of biocompatible materials. Thus for example biocompatible thread can be used to stitch the handling gauze 16 to the reinforcing gauze 10. Also for example a biocompatible glue can be used to glue the handling gauze 16 to the reinforcing gauze 10. Also for example biocompatible staples can be used to connect the handling gauze 16 with the reinforcing gauze 10. Further means which are known by the expert can also be used.

By attaching the flaps 14 to the handling gauze 16 at the site of the edge 17, it is achieved that in its position when placed in tubular form through the reinforcing gauze 10 as shown in FIG. 2, the tubular handling gauze 16 protrudes to a limited extent on the ingrowth side of the reinforcing gauze 10, this protrusion distance being indicated in FIG. 2 by indication H1. Thus the site of the edge 17 can be interpreted as a distance from the edge, which distance is preferably less than 15 mm, more preferably less than 10 mm and preferably greater than 1 mm, more preferably greater than 2 mm and most preferably greater than 5 mm. Preferably the handling gauze 16 is formed such that in the position described above, the handling gauze extends at least as far on the intestinal side 12 of the reinforcing gauze 10 as on the ingrowth side 11. The distance by which the reinforcing gauze extends on the intestinal side is illustrated in FIG. 2 with indication H2. Preferably distance H2 is greater than distance H1, more preferably distance H2 is greater than twice the distance H1, most preferably distance H2 is greater than three times distance H1. Thus the handling gauze is formed so that a length L measured perpendicular to said straight edge 17 is preferably greater than 1.5 cm, more preferably greater than 2.5 cm, most preferably greater than 3.5 cm.

A first flap 14 adjacent to the stoma access path 15 is indicated in FIG. 1 by the flap marked A. The second flap 14 adjacent to the stoma access path 15 is shown in FIG. 1 as flap marked F. Between the flap marked A and the flap marked F, the stoma access path 15 makes a connection with the opening 13, wherein these flaps marked A and F are regarded as adjacent to said stoma access path 15. The flap marked B is not for example regarded as adjacent to the stoma access path 15 since the flap marked A sits between the flap marked B and the stoma access path 14.

Preferably the first flap 14 adjacent to the stoma access path 15 is connected with the handling gauze 16 at the site of the further end 19 of the straight edge 17. This results in the stoma access path 15 in the reinforcing gauze 10 being aligned with the access to the tubular positioned handling gauze 16 as illustrated in FIG. 4 with arrow 20. Thus the gauze assembly according to the invention can be placed around a bowel. Furthermore it is also possible to hold the stoma access path 15 open together with the tubular positioned handling gauze 16, which is an advantage when positioning such a gauze assembly as will be explained below.

Preferably the second flap 14 adjacent to the stoma access path 15 is connected with the handling gauze 16 at a distance K from the edge 18. This distance K is preferably greater than 2 mm, more preferably greater than 7 mm, most preferably greater than 12 mm. This distance K results, on tubular positioning of the handling gauze 16 through the reinforcing gauze 10, in an overlap with the handling part. This overlap is shown in FIG. 4 with indication M. The overlap M makes it possible to hold the handling gauze 16 in a tubular positioned site by connecting the overlapping parts with each other in this position. Furthermore the overlap M simplifies handling of the gauze assembly because this overlap is positioned in a location where the gauze assembly 16 can be glued in place, as will be explained below.

It will be clear from the description above that the end 18 and the further end 19 of said straight edge 17 can be reversed without deviating from the principles of the invention. Also the first flap 14 marked A and the second flap 14 marked F in the description can be reversed without significant differences in function and effect of the gauze assembly according to the invention. Therefore it will be clear that the invention and the interpretation of the claims is not limited to the embodiment according to the specific description above.

A gauze assembly according to the invention is preferably positioned laparoscopically. This has a number of features for which the gauze assembly according to the invention is adapted. In laparoscopic intervention, an operation is carried out with minimum cutting. On a laparoscopic intervention in the abdomen, the abdomen is inflated with gas whereby in the abdomen a working cavity 21 is created. The operation is performed using a camera which is introduced into the working cavity 21 in the inflated abdomen and using tools such as clamps which are introduced into the working cavity 21 and can be operated from outside the abdomen. This way of working greatly reduces the visibility and ability to be handled of items inside the working cavity 21. The gauze assembly according to the invention is adapted to this limited visibility and handling ability.

When positioning a gauze assembly according to the invention, the gauze assembly is introduced into the working cavity 21. Then the gauze assembly is roughly positioned around the bowel 1 which extends through the abdominal wall, with the ingrowth side of the gauze assembly facing towards the abdominal wall and the intestinal side of the gauze assembly facing towards the bowel. To position the gauze assembly roughly around the bowel 1, the gauze assembly is moved in relation to the bowel so that the bowel follows the arrow shown in FIG. 4. In this position both the reinforcing gauze 10 and the handling gauze 16 substantially extend around the bowel 1.

Furthermore the gauze assembly must be finely positioned in relation to the bowel 1 and the abdominal wall 2 and attached in this position to the bowel 1 and the abdominal wall 2. Such a finely positioned gauze assembly is shown in FIG. 3. In this position firstly the place of the reinforcing gauze 10 in relation to the abdominal wall 2 and secondly the place of the handling gauze 16 in relation to the bowel 1 are established. Thus the opening 8 shown in FIG. 5 is drawn tightly to achieve a correct position of the reinforcing gauze 10 in relation to the abdominal wall 2. Furthermore the handling gauze 16 is positioned in relation to the bowel 1 so that a correct length of the bowel 1 lies between firstly the adhesion with the abdominal wall 4 and secondly an inside of the muscle tissue 6. This correct length of bowel 1 is substantially equal to or preferably slightly less than the thickness of the abdominal wall 2.

Such a fine positioning is typically obtained by holding the stoma access path 15 open in the first phase. This allows, at the site of the opened stoma access path 15, a view behind the gauze assembly and gripping using gripper means. Thus fine positioning and attachment of the flaps 14 can be obtained in relation to the bowel 1. Through the opened stoma access path 15 the handling gauze 16 is gripped firstly with gripping means at the site of the end 18 and secondly with further gripping means at the site of the further end 19. These gripping means can be moved firstly so that the end 18 overlaps with the further end 19 as shown in FIG. 4 and secondly shifted in relation to the bowel 1 so that the handling gauze 16 in relation to the bowel 1 assumes a correct position as shown in FIG. 3. In this position wherein the stoma access path 15 is still held open to give access to elements behind the reinforcing gauze 10, the overlapping parts are connected together and the handling gauze, preferably at the point of the overlapping parts, is connected with the bowel 1. These connections can be achieved in various ways known to the expert. By connecting the overlapping parts with each other and by connecting the handling gauze at least at one point with the bowel 1 at the straight edge 17, all flaps 14 are fixed in the correct manner and correct position to the bowel 1. Namely by the latter connections the straight edge 17 of the handling gauze 16 is correctly connected with the bowel 1 while the flaps 14 are connected with the straight edge 17 and hence also correctly positioned and connected in relation to the bowel 1. This achieves in a simple manner what was impossible in the prior art, namely correct positioning and fixing of the flaps 14 on the bowel 1 viewed from the working cavity 21 behind the plane in which the reinforcing gauze 10 substantially extends.

In a second phase the stoma access path 15 is drawn tightly such that the reinforcing gauze 10 lies correctly in relation to the abdominal wall 2 as shown in FIG. 3. Drawing the stoma access path 15 tightly has the result that everything which, viewed from the working cavity 21, lies behind the reinforcing gauze 10 becomes invisible and inaccessible. However in this second phase the flaps 14 and the handling gauze 16 are already correctly positioned and connected with the bowel 1 so that these elements behind the reinforcing gauze 10 need no longer be visible and accessible. The reinforcing gauze 10 and the part of the handling gauze 16 which lies on the intestinal side 12 of the reinforcing gauze 10 in the second phase are connected respectively with the abdominal wall 2, in particular the muscle tissue 6, and the bowel 1. This positioning and connecting in the second phase does not constitute a substantial problem as all elements to be positioned and connected are visible from the working cavity 21.

The positioned gauze assembly according to the invention has a further advantage of minimising future recurrence, i.e. a new parastomal hernia after the present parastomal hernia has been repaired. FIG. 3, viewed at the level of the muscle tissue 6 and the centre 3 of the bowel 1 in a direction perpendicular to the bowel, shows a succession of the following elements: bowel tissue 1, handling gauze 16, intestinal side of flap 14, ingrowth side of flap 14, and muscle tissue. The intestinal side of the flap 14 is intended, if in contact with tissue, to not grow into this tissue and to irritate the tissue as little as possible. However the intestinal side of the flap 14 in the above succession does not lie directly against the bowel tissue 1 but against the handling gauze 16 which in turn lies against the bowel tissue. As a result the handling gauze 16 causes irritation of the bowel tissue and thus the growth of scar tissue is stimulated locally while the handling gauze 16 is connected with the flap 14. The flap 14 on its ingrowth side grows into the muscle tissue 6. However by the formation of scar tissue on the flap 14 at the side of the bowel 1, a tissue connection occurs between the bowel 1 and flap 14 and/or muscle tissue 6. This tissue connection prevents the occurrence of a future parastomal hernia.

Because this tissue connection in a preferred embodiment occurs at the site of the flap ends, a diameter variation of the bowel 1 can still be absorbed. This can occur by dilation or stretching open the scar tissue which extends between adjacent flaps, whereby the flap ends will fold towards the outside, or a contraction whereby the flap ends will fold more towards the inside.

1. Gauze assembly for reinforcing tissue which extends around a surgically created stoma, wherein said gauze assembly comprises a biocompatible reinforcing gauze with a first side and a second side such that said reinforcing gauze can grow into said tissue, which reinforcing gauze in a central zone has a stoma opening, which stoma opening has an edge formed by flaps which extend from said reinforcing gauze substantially towards a centre point of said stoma opening, which reinforcing gauze furthermore has a stoma access path which extends from said stoma opening to an outside edge of the reinforcing gauze, characterised in that said gauze assembly furthermore comprises a biocompatible handling gauze, which handling gauze is connected with the second side of said flaps such that said handling gauze is positionable in a substantially tubular form through said stoma opening and said flaps can be folded at least partly in the direction of said first side.

2. Gauze assembly according to claim 1, wherein said first side is an ingrowth side and said second side an intestinal side.

3. Gauze assembly according to claim 1, wherein said handling gauze has at least one substantially straight edge, said flaps being connected with said handling gauze at the said edge.

4. Gauze assembly according to claim 3, wherein a first flap of said flaps adjacent to said stoma access path is connected at an end of said straight edge.

5. Gauze assembly according to claim 4, wherein a second flap of said flaps adjacent to said stoma access path is connected at a distance from another end opposite the said end of said straight edge such that on tubular positioning of said handling gauze, an overlap of the handling gauze occurs.

6. Gauze assembly according to claim 1, wherein each of said flaps has a flap end which lies closest to said centre point, said flaps being connected with said handling gauze at their flap ends.

7. Gauze assembly according to claim 1, wherein said handling gauze is made from a material which is fully decomposable by tissue.

8. Gauze assembly according to any of the preceding claims claim 1, wherein said flaps are formed sawtooth-like.

9. Set of a biocompatible reinforcing gauze and a biocompatible handling gauze intended for production of a gauze assembly according to claim 1.
